(19) United States
(12) Patent Application Publication
(10) Pub. No.: US 2008/0306131 A1
(43) Pub. Date: Dec. 11, 2008

(54) Progesterone receptor modulator and uses thereof

(75) Inventors: Laurel Rita Barbieri, Blauvelt, NY (US); Thomas Joseph Berrodin, Collegeville, PA (US); Ramunas Bigelis, Congers, NY (US); Li-Ping Chang, Spring Valley, NY (US); Deborah Marie Roll, Nanuet, NY (US); Maya Prakash Singh, Bardonia, NY (US); Matthew R. Yudt, Schwenksville, PA (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/156,504

(22) Filed: Jun. 2, 2008

Related U.S. Application Data
(60) Provisional application No. 60/933,292, filed on Jun. 5, 2007.

(51) Int. Cl. A61K 31/403; C07D 209/56; C12P 17/10; A61P 25/00; C12N 1/14

(52) U.S. Cl. 514/410; 548/418; 435/121; 435/254.1

(57) ABSTRACT

Compounds of the structure:

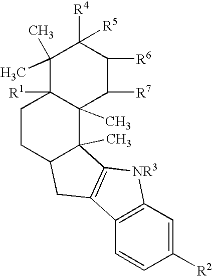

are provided. Also provided are methods of using these compounds as progesterone receptor modulators. Also described are methods of producing these compounds from a Verticillium species.

### CROSS-REFERENCE TO RELATED APPLICATION

This application claims the benefit under 35 USC 119(e) of U.S. Provisional Patent Application No. 60/933,292, filed Jun. 5, 2007.

### BACKGROUND OF THE INVENTION

This invention relates to agonists and antagonists of the progesterone receptor, their preparation and utility.

Nuclear hormone receptors (NHR) form a class of structurally related gene regulators known as “ligand dependent transcription factors” (Mangelsdorf, D. J. et al. Cell, 83, 835, 1995). The steroid receptor family is a subset of the intracellular receptor (IR) family, including progesterone receptor (PR), estrogen receptor (ER), androgen receptor (AR), glucocorticoid receptor (GR), and mineralocorticoid receptor (MR).

The natural hormone, or ligand, for the PR is the steroid progesterone, but synthetic compounds, such as medroxyprogesterone acetate or levonorgestrel, have been made which also serve as PR ligands. Once a ligand is present in the fluid surrounding a cell, it passes through the membrane via passive diffusion, and binds to the IR/NHR to create a receptor/ligand complex. This complex binds to specific gene promoters present in the cell's DNA. Once bound to the DNA the complex modulates the production of mRNA and the protein encoded by that gene.

A compound that binds to an IR/NHR and mimics the action of the natural hormone is termed an agonist, whilst a compound that inhibits the effect of the hormone is an antagonist. Many ligands to NHRs have been demonstrated to exhibit partial or selective agonism or antagonism, often in cell- or tissue-selective fashion, and have henceforth been called NHR “modulators” as a broad term to cover these partial and tissue selective activities observed.

PR agonists (natural and synthetic) are known to play an important role in the health of women. PR agonists are used in birth control formulations, either along with or in the presence of an ER agonist. ER agonists are used to treat the symptoms of menopause, but have been associated with a proliferative effect on the uterus that can lead to an increased risk of uterine cancers. Co-administration of a PR agonist reduces/ablates that risk.

PR antagonists may also be used in contraception. In this context they may be administered alone (Ulmann, et al., Ann. N.Y. Acad. Sci., 261, 248, 1995), in combination with a PR agonist (Kekkonen, et al., Fertility and Sterility, 60, 610, 1993) or in combination with a partial ER antagonist such as tamoxifen (U.S. Pat. No. 5,719,136).

PR antagonists may also be useful for the treatment of hormone dependent breast cancers (Horwitz, et al., Horm. Cancer, 283, 1996, pub: Birkhaeuser, Boston, Mass., ed. Vedeckis) as well as uterine and ovarian cancers. PR antagonists may also be useful for the treatment of non-malignant chronic conditions such as uterine fibroids (Murphy, et al., J. Clin. Endo. Metab., 76, 513, 1993) and endometriosis (Kettel, et al., Fertility and Sterility, 56, 402, 1991). PR antagonists may also be useful in hormone replacement therapy for postmenopausal patients in combination with a partial ER antagonist such as tamoxifen (U.S. Pat. No. 5,719,136).

PR antagonists, such as mifepristone and onapristone, have been shown to be effective in a model of hormone dependent prostate cancer, which may indicate their utility in the treatment of this condition in men (Michna, et al., Ann. N.Y. Acad. Sci., 761, 224, 1995).

### SUMMARY OF THE INVENTION

In one aspect, the invention provides a compound having the structure:

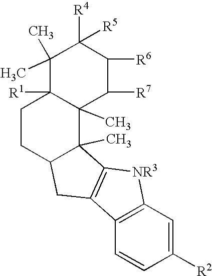

wherein R¹ is H or OH;

### R² is H or

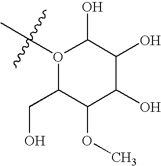

### R³ is H or CH₃;

### R⁴, R⁵ is ═O, OH;

R⁶, R⁷ is CH═CH, CH₂—CH₂, OH, and an epoxide;
or a pharmaceutically acceptable salt, ester, or ether thereof.

In another aspect, the invention provides an isolated culture of Verticillium lecanii strain ARSEF 6144-A, assigned Agricultural Research Service Culture Collection (NRRL) Accession No. NRRL 30907 (originally deposited under the name MTA6144-A, but renamed to conform to naming conventions).

In still a further aspect, the invention provides a process for the preparation of a compound of the invention. The method comprises culturing a strain of Verticillium lecanii capable of producing the compound, and isolating the compound.

In yet another embodiment, the invention provides a progesterone receptor modulator composition comprising a compound of the invention and a pharmaceutically acceptable carrier or excipient.

In still another embodiment, the invention provides a method of inducing contraception, providing hormone replacement therapy, treating cycle-related symptoms, and treating or preventing benign or malignant neoplastic disease comprising administering to a mammal in need thereof a pharmaceutically effective amount of a compound of the invention.

In yet a further embodiment, the invention provides a method of treating neurodegenerative disorders comprising administering to a mammal in need thereof a pharmaceutically effective amount of a compound of the invention.

Other aspects and advantages of the invention will be readily apparent from the following detailed description of the invention.

### DETAILED DESCRIPTION OF THE INVENTION

The present invention provides a compound having the structure I:

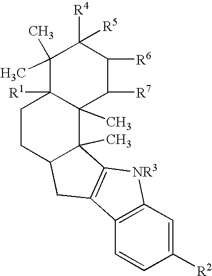

wherein R¹ is H or OH;

### R² is H or

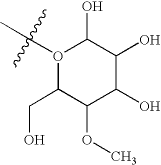

### R³ is H or CH₃;

### R⁴, R⁵ is ═O, OH;

R⁶, R⁷ is CH═CH, CH₂—CH₂, OH, and an epoxide;
or a pharmaceutically acceptable salt, ester, or ether thereof.

The novel compounds of this invention have been shown to be active in a progesterone receptor agonist screen and to act as PR modulators in functional models. These PR modulators are thereby effective as PR agonists or PR antagonists.

As used herein, the terms “anti-progestational agent”, “anti-progestin” and “progesterone receptor antagonist” are understood to be synonymous. Similarly, “progestin”, “progestational agent”, and “progesterone receptor agonist” are understood to refer to compounds of the same activity.

Thus, in one aspect, the invention provides a compound of formula I, which is useful as progesterone receptor (PR) modulators and in treating a variety of conditions. Nonsteroidal PR agonists such as are described herein have use in female contraceptives and hormone therapy regimens, as well as in other pharmaceutical applications.

In one embodiment, a PR modulator compound of the invention is characterized by the following structure:

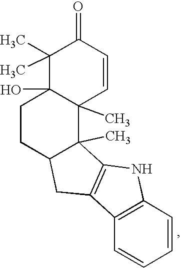

or a salt or prodrug thereof. In one embodiment, the compound 4a-hydroxy-4,4,12b,12c-tetramethyl-4a,5,6,6a,7,12,12b,12c-octahydrobenzo[6,7]indeno[1,2-b]indol-3(4H)-one, or a salt or prodrug thereof, is characterized by one or more of the following:
a) Molecular Formula: C₂₃H₂₇NO₂
b) Molecular Weight: high resolution positive ion electrospray MS m/z=350.21161 (M+H)⁺; high resolution negative ion electrospray MS m/z=348.19630 (M−H)⁻
c) Ultraviolet Absorption Spectrum: λₘₐₓ nm (acetonitrile-water-formic acid)=234, 284
d) Proton NMR (400 MHz; δ, CDCl₃): 7.92, 7.49, 7.35, 7.31, 7.12, 7.11, 6.18, 2.85, 2.77, 2.43, 2.07, 1.96, 1.84, 1.80, 1.51, 1.40, 1.32, 1.25;
e) Carbon NMR (100 MHz; δ, CDCl₃): 203.9, 155.2, 152.3, 140.3, 128.4, 125.4, 121.1, 120.1, 118.9, 118.1, 111.8, 80.4, 53.4, 52.3, 49.6, 48.2, 29.7, 28.4, 27.5, 25.6, 23.7, 21.4, 19.8.

In another embodiment, a PR modulator compound of the invention has the structure:

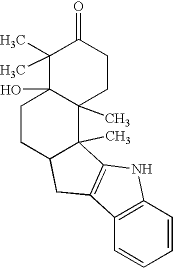

or a salt or prodrug thereof. In one embodiment, the compound 4a-hydroxy-4,4,12b,12c-tetramethyl-1,4,4a,5,6,6a,7,12,12b,12c-decahydrobenzo[6,7]indeno[1,2-b]indol-3(2H)-one, or a salt or prodrug thereof, is characterized by one or more of the following:
a) Molecular Formula: C₂₃H₂₉NO₂
b) Molecular Weight: high resolution positive ion electrospray MS m/z 352.22700 (M+H)⁺; high resolution negative ion electrospray MS m/z=350.21242 (M−H)⁻
c) Ultraviolet Absorption Spectrum: λₘₐₓ nm (acetonitrile-water-formic acid)=232, 284;
d) Proton NMR (400 MHz; δ, CHCl₃): 7.73, 7.44, 7.31, 7.09, 7.08, 2.92, 2.82, 2.75, 2.74, 2.72, 2.42, 2.03, 1.97, 1.73, 1.65, 1.63, 1.34, 1.27, 1.25, 1.13;
e) Carbon NMR (100 MHz; 5, CDCl₃): 216.8, 152.7, 140.2, 125.4, 120.8, 119.9, 118.8, 117.4, 111.7, 81.5, 54.9, 53.3, 49.5, 43.7, 34.7, 32.5, 29.9, 27.7, 24.5, 23.1, 22.6, 21.3, 17.0.

In another embodiment, a PR modulator compound has the structure:

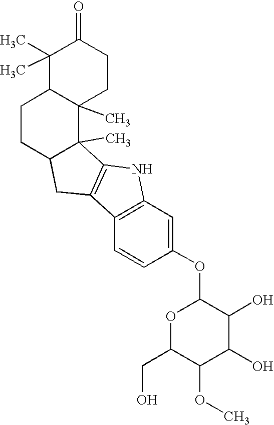

or a salt or prodrug thereof. In one embodiment, the compound (4aS,6aR,12bR,12cR)-4,4,12b,12c-tetramethyl-3-oxo-3,4,4a,5,6,6a,7,12,12b,12c-decahydrobenzo[6,7] indeno[1,2-b]indol-10-yl 4-O-methyl-α-D-glucopyranoside, or a salt or prodrug thereof, is characterized by one or more of the following:

a) Molecular Formula: C₃₀H₃₉NO₇;

b) Molecular Weight: positive ion electrospray MS m/z=526.2 (M+H)⁺; high resolution FTMS m/z=525.26514 (M)⁺;

c) Ultraviolet Absorption Spectrum: λₘₐₓ nm (acetonitrile-water-formic acid)=236, 278, 300;

d) Proton NMR (400 MHz; δ, CDCl₃): 8.01, 7.33, 7.24, 7.07, 6.86, 6.03, 4.88, 3.94, 3.77, 3.75, 3.68, 3.62, 3.44, 3.30, 2.76, 2.31, 1.87, 1.80, 1.71, 1.56, 1.32, 1.26, 1.22, 1.12, 1.07.

In yet another embodiment, the invention provides a compound having the structure:

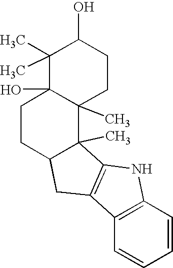

or a salt or prodrug thereof. In one embodiment, the compound (3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol, or a salt or prodrug thereof, is characterized by one or more of the following:

a) Molecular Formula: C₂₃H₃₁NO₂

b) Molecular Weight: high resolution ESI FTMS m/z=354.24264 (M+H)⁺; high resolution ESI FTMS m/z=352.22817 (M−H)⁻;

c) Ultraviolet Absorption Spectrum: λₘₐₓ nm (acetonitrile-water-formic acid)=234, 284

d) Proton NMR (400 MHz; δ, CDCl₃): 7.72, 7.43, 7.30, 7.08, 7.07, 3.62, 2.84, 2.69, 2.66, 2.38, 2.30, 2.00, 1.87, 1.74, 1.72, 1.68, 1.38, 1.25, 1.24, 1.14, 1.03;

e) Carbon NMR (100 MHz; δ, CDCl₃): 154.3, 139.9, 125.6, 120.4, 119.7, 119.6, 117.3, 111.6, 81.5, 78.5, 54.9, 49.6, 43.9, 42.4, 30.5, 27.7, 26.7, 25.3, 24.8, 24.4, 23.4, 21.9, 17.0.

The compounds of the present invention can be used in the form of salts derived from pharmaceutically or physiologically acceptable acids or bases. These salts include, but are not limited to, the following salts with organic and inorganic acids such as acetic, lactic, citric, tartaric, succinic, fumaric, maleic, malonic, mandelic, malic, hydrochloric, hydrobromic, phosphoric, nitric, sulfuric, methanesulfonic, toluenesulfonic and similarly known acceptable acids, and mixtures thereof. Other salts include salts with alkali metals or alkaline earth metals, such as sodium (e.g., sodium hydroxide), potassium (e.g., potassium hydroxide), calcium or magnesium.

These salts, as well as other compounds produced by the method of the invention may be in the form of esters, carbamates and other conventional “pro-drug” forms, which, when administered in such form, convert to the active moiety in vivo. In one desirable embodiment, the prodrugs are esters. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

As described herein, the compounds of the invention and/or salts, prodrugs, or tautomers thereof, are delivered in regimens therapeutic or prophylactic purposes, as described herein.

The compounds discussed herein also encompass “metabolites” which are unique products formed by processing the compounds of the invention by the cell or subject. Desirably, metabolites are formed in vivo.

II. Verticillium lecanii Strains

In one aspect, the present invention provides a method of culturing a Verticillium species in order to produce a compound of the invention.

In one embodiment, a culture of entomopathogenic Verticillium lecanii was used to produce a nonsteroidal compound of the invention. However, the culture showed instability and variability, especially after subculturing on agar media, as has been reported in the literature for other strains of V. lecanii. A yellow-pigmented morphological variant (variant A) with reduced aerial mycelia was identified on malt extract agar, and shown to produce an active product with consistency. The other 3 variants of V. lecanii were less productive in all media investigated.

Thus, in another embodiment, the invention provides an isolated culture of variant strain A. This culture facilitated medium development and optimization, as well as reliable production of both the bioactive target compound and also another analog that can be chemically converted to the active form. Production medium containing sucrose, glycerol, yeast extract, calcium carbonate, and ammonium sulfate was formulated, and nitrogen levels were optimized for production of an active PR modulatory compound at 22° C. Ammonium sulfate levels greater than 7 g/L repressed production of 4 metabolites, while levels of 4.6-7 g/L were associated with favorable yields of a combination of a PR modulator and its analog.

Wyeth strain ARSEF 6144-A refers to a strain of Verticillium sp. that has been deposited under the terms of the Budapest Treaty with the Agricultural Research Service Culture Collection (NRRL), 1815 North University Street, Peoria, Ill. 61604 USA on Mar. 14, 2006 (Accession No. NRRL 30907). [It is noted that The ARSEF classification of all entomopathogenic species in Verticillium is currently reassigning these fungi to a series of other genera, primarily Lecanicillium whose type species is Lecanicillium lecanii (R. A. Humber, Curator, ARSEF, US Plant, Soil & Nutrition Laboratory, Ithaca, N.Y.). Therefore, ARSEF isolates of Verticillium lecanii will reidentified following the segregation of this widespread, common species.

Fermentation conditions to culture the Verticillium species described herein can be performed in flasks. Alternatively, production of higher volumes can be performed in fermentors under similar conditions.

Media useful for the cultivation of Verticillium species and the production of the macrolide compounds include assimilable carbon sources such as, for example, dextrose, sucrose, glycerol, molasses, starch, galactose, fructose, corn starch, malt extract and combinations thereof; an assimilable source of nitrogen such as, for example, ammonium chloride, ammonium sulfate, ammonium nitrate, sodium nitrate, amino acids, protein hydrolysates, corn steep liquor, casamino acids, yeast extract, peptone, tryptone and combinations thereof; and inorganic anions and cations such as, for example, potassium, sodium, sulfate, calcium, magnesium, chloride. Trace elements such as, for example, zinc, cobalt, iron, boron, molybdenum, and copper are supplied as impurities of other constituents of the media. Aeration in tanks and bottles is supplied by forcing sterile air through or onto the surface of the fermenting medium. A mechanical impeller provides further agitation in tanks. An antifoam agent such as polypropylene glycol can be added as needed.

For example, see Sigma Aldrich (St. Louis, Mo.); G. J. Tortora et al, Microbiology: An Introduction Media Update (Benjamin Cummings Publishing Co; Oct. 1, 2001); Maintaining Cultures for Biotechnology and Industry, eds. J.C. Hunter-Cevera and A. Bet (Academic Press, Jan. 25, 1996).

After about 5 to 10 days, and preferably about 7 days of fermentation at a temperature in the range of about 22° C. to 28° C., the cells from the culture are pelleted by centrifugation. In one embodiment, the cells are extracted with a suitable solvent, e.g., ethyl acetate. The extract is concentrated in vacuo and resuspended in a minimum volume of a suitable solvent, e.g., methanol. The solution is loaded onto a reverse phase silica column and eluted with 20%-100% methanol in water. The fractions eluting from 60% methanol to 100% methanol are concentrated in vacuo. The analog(s) containing fractions are separated by suitable means, e.g., chromatographic methods.

In another embodiment, the supernatant is mixed with a suitable resin and stirred for about 2 to 16 hours. Thereafter, the resin is washed with a suitable solvent, e.g., methanol, and the filtrate collected. To the cell pellet, an ethyl acetate-methanol mixture is added. This is repeatedly shaken and centrifuged, and the supernatant collected. The cell supernatant and the broth methanol filtrate are combined and concentrated in vacuo. Crude extract is adsorbed onto silica, and fractionated by vacuum liquid chromatography (VLC). The compound is eluted with a suitable solvent, e.g., methanol in dichloromethane. This extract is concentrated, adsorbed onto silica and loaded onto a flash silica column. The compound is eluted with a suitable solvent, concentrated and further purified by column chromatography.

Enzymes of the present invention can be recovered and purified from cell cultures by well-known methods including ammonium sulfate or ethanol precipitation, acid extraction, anion or cation exchange chromatography, phosphocellulose chromatography, hydrophobic interaction chromatography, high performance liquid chromatography, hydroxylapatite chromatography and lectin chromatography. Most preferably, affinity chromatography is employed for purification. Well-known techniques for refolding proteins may be employed to regenerate active conformation when the enzyme is denatured during isolation and or purification.

The presence of a compound produced by the organism in the crude or semi-purified material can be confirmed by conventional methods, e.g., liquid chromatography mass spectrometric (LCMS) analysis of fractions. These fractions may be pooled and further purified by chromatographic methods, and optionally concentrated, e.g., in vacuo.

The resulting purified compounds are free of cells and cellular materials, by-products, reagents, and other foreign material as necessary to permit handling and formulating of the compound for laboratory and/or clinical purposes. It is preferable that purity of the compounds used in the present invention have a purity of greater than 80% by weight; more preferably at least 90% by weight, even more preferably greater than 95% by weight; yet even more preferably at least 99% by weight. In one embodiment, the invention provides compositions containing the compounds of the invention, regardless of how such compounds are produced.

In yet another embodiment, the invention provides a novel compound produced by the isolated Verticillium strain described herein.

### II. Pharmaceutical Compositions

This invention also includes pharmaceutical compositions containing one or more compounds of this invention and a pharmaceutically acceptable carrier or excipient. The invention also includes methods of treatment which include administering to a mammal a pharmaceutically effective amount of one or more compounds as described as progesterone receptor modulators.

The compounds of the invention may be combined with one or more pharmaceutically acceptable carriers or excipients, for example, solvents, diluents and the like. Suitably, the compounds of the invention are formulated for delivery to a subject by any suitable route including, e.g., transdermal, mucosal (intranasal, buccal, vaginal), oral, parenteral, among others. A variety of suitable delivery devices can be utilized and include, without limitation, tablets, caplets, capsules, gel tabs, dispersible powders, granules, suspensions, injectable solutions, transdermal patches, topical creams or gels, and vaginal rings, among others.

One particularly desirable pharmaceutical composition, from the standpoint of ease of preparation and administration, are solid compositions, particularly tablets and hard-filled or liquid-filled capsules. Oral administration of the compounds is most desirable.

The compounds of the invention may be administered orally as well as by intravenous, intramuscular, or subcutaneous routes. Solid carriers include starch, lactose, dicalcium phosphate, microcrystalline cellulose, sucrose and kaolin. Liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, butylatedhydroxytoluene (BHT) and butylatedhydroxyanisole (BHA).

When formulated for oral delivery, the compound of the invention can be in the form of a tablet, capsule, caplet, gel tab, dispersible powders, granules, or suspensions. In one embodiment, the compound can be combined with suspending agents, including about 0.05 to about 5% of suspending agent, syrups containing, for example, about 10 to about 50% of sugar, and/or elixirs containing, for example, about 20 to about 50% ethanol, and the like.

The compounds of the invention may also be administered parenterally or intraperitoneally. Solutions or suspensions of the compounds of the invention as a free base or pharmacologically acceptable salt can be prepared in water suitably mixed with a surfactant such as hydroxypropylcellulose. Dispersions can also be prepared in glycerol, liquid, polyethylene glycols and mixtures thereof in oils. In one embodiment, the solutions or suspensions containing the compound of the invention can contain about 0.05 to about 5% suspending agent in an isotonic medium. Such pharmaceutical preparations may contain, for example, about 25 to about 90% of the compound in combination with the carrier. Desirably, the pharmaceutical preparation contains about 5% and 60% by weight of the compound.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringe ability exits. It must be stable under conditions of manufacture and storage and must be preserved against the contaminating action of microorganisms such as bacterial and fungi. The carrier can be a solvent or dispersion medium containing, for example, water, ethanol (e.g., glycerol, propylene glycol and liquid polyethylene glycol), suitable mixtures thereof, and vegetable oil.

The compounds of the invention may also be administered via a vaginal ring. Suitably, use of the vaginal ring is timed to cycle to which the compound of the invention is being administered, including a 28-day cycle. However, the vaginal ring can be inserted for longer or shorter periods of time. See, U.S. Pat. Nos. 5,972,372; 6,126,958; and 6,125,850, which are hereby incorporated by reference, for formulations of the vaginal ring that can be used in the present invention.

The compound of the invention can also be delivered via a transdermal patch. Suitably, use of the patch is timed to the length of the cycle, including a 28-day cycle. However, the patch can remain in place for longer or shorter periods of time.

These compounds can be utilized in methods of contraception, hormone replacement therapy, and the treatment and/or prevention of benign and malignant neoplastic disease, cycle-related symptoms, uterine myometrial fibroids, endometriosis, benign prostatic hypertrophy; carcinomas and adenocarcinomas of the endometrium, ovary, breast, colon, prostate, pituitary, meningioma and other hormone-dependent tumors, dysmenorrheal, dysfunctional uterine bleeding, symptoms of premenstrual syndrome and premenstrual dysphoric disorder, and for inducing amenorrhea. Additional uses of the present progesterone receptor modulators include the synchronization of estrus in livestock. In one embodiment, the neoplastic disease is hormone-dependent.

The term “cycle-related symptoms” as used herein refers to psychological and physical symptoms associated with a woman's menstrual cycle arising in the luteal phase of the menstrual cycle. It has been reported that most women report experiencing cycle-related symptoms. The symptoms generally disappear after the onset of menstruation, and the patient is free from symptoms during the rest of the follicular phase. The cyclical nature of the symptom variations is characteristic of cycle-related symptoms.

Cycle-related symptoms occur in about 95% of women who experience some physical or mood changes with their menstrual cycles. Only about one-third of those women experiences moderate to severe cycle-related symptoms. Women vary in the number, type, severity, and pattern of symptoms before menstruation. One thing common to all the types of cyclic-related symptoms is the decrease or elimination of the symptoms in the two weeks after menstruation up to ovulation.

The term “cycle-related symptoms” refers to psychological symptoms (for example, mood change, irritability, anxiety, lack of concentration, or decrease in sexual desire) and physical symptoms (for example, dysmenorrhea, breast tenderness, bloating, fatigue, or food cravings) associated with a woman's menstrual cycle. Cycle-related symptoms occur after ovulation but before menses and usually terminate at the start of the menstrual period or shortly thereafter. Cycle-related symptoms include, but are not limited to, dysmenorrhea and moderate to severe cycle-related symptoms.

In another embodiment, the compounds of the invention are useful in treating and/or alleviating symptoms of neurodegenerative disorders. Examples of such neurodegenerative disorders include those selected from the group consisting of Alzheimer's disease, Parkinson's disease, and neuronal damage following ischemia or trauma. Suitable assays for determining the neuroactivity effect of progesterone receptor modulators have been described. See, e.g., A. M. Ghoumari, et al, J Neurochem, 2003, 86, 848-859 and E.-E. Baulieu and M. Schumacher, Steroids, 65 (2000), 605-612.

In one embodiment, the compounds of the invention can be administered in combination with other agents, as well as in combination with each other. Such agents include, without limitation, progestins, antiprogestins, estrogens, among others. Progestins can include, without limitation, tanaproget, levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, (17-deacetyl)norgestimate. Estrogens can include, without limitation, ethinyl estradiol. Antiprogestins can include, without limitation, mifepristone {RU-486} and onapristone {ZK-98,299}.

A patient or subject being treated is a mammalian subject and typically a female. Desirably, the subject is a human. However, as used herein, a female can include non-human mammals, e.g., cattle or livestock, horses, pigs, domestic animals, etc.

In one embodiment, also includes pharmaceutical compositions containing one or more compounds of this invention and a pharmaceutically acceptable carrier or excipient. The invention also includes methods of treatment which include administering to a mammal a pharmaceutically effective amount of one or more compounds as described as progesterone receptor modulators.

The compounds of the invention may be combined with one or more pharmaceutically acceptable carriers or excipients, for example, solvents, diluents and the like. Suitably, the compounds of the invention are formulated for delivery to a subject by any suitable route including, e.g., transdermal, mucosal (intranasal, buccal, vaginal), oral, parenteral, among others. A variety of suitable delivery devices can be utilized and include, without limitation, tablets, caplets, capsules, gel tabs, dispersible powders, granules, suspensions, injectable solutions, transdermal patches, topical creams or gels, and vaginal rings, among others.

One particularly desirable pharmaceutical composition, from the standpoint of ease of preparation and administration, are solid compositions, particularly tablets and hard-filled or liquid-filled capsules. Oral administration of the compounds is most desirable.

The compounds of the invention may be administered orally as well as by intravenous, intramuscular, or subcutaneous routes. Solid carriers include starch, lactose, dicalcium phosphate, microcrystalline cellulose, sucrose and kaolin. Liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, butylatedhydroxytoluene (BHT) and butylatedhydroxyanisole (BHA).

When formulated for oral delivery, the compound of the invention can be in the form of a tablet, capsule, caplet, gel tab, dispersible powders, granules, or suspensions. In one embodiment, the compound can be combined with suspending agents, including about 0.05 to about 5% of suspending agent, syrups containing, for example, about 10 to about 50% of sugar, and/or elixirs containing, for example, about 20 to about 50% ethanol, and the like.

The compounds of the invention may also be administered parenterally or intraperitoneally. Solutions or suspensions of the compounds of the invention as a free base or pharmacologically acceptable salt can be prepared in water suitably mixed with a surfactant such as hydroxypropylcellulose. Dispersions can also be prepared in glycerol, liquid, polyethylene glycols and mixtures thereof in oils. In one embodiment, the solutions or suspensions containing the compound of the invention can contain about 0.05 to about 5% suspending agent in an isotonic medium. Such pharmaceutical preparations may contain, for example, about 25 to about 90% of the compound in combination with the carrier. Desirably, the pharmaceutical preparation contains about 5% and 60% by weight of the compound.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringe ability exits. It must be stable under conditions of manufacture and storage and must be preserved against the contaminating action of microorganisms such as bacteria and fungi. The carrier can be a solvent or dispersion medium containing, for example, water, ethanol (e.g., glycerol, propylene glycol and liquid polyethylene glycol), suitable mixtures thereof, and vegetable oil.

The compounds of the invention may also be administered via a vaginal ring. Suitably, use of the vaginal ring is timed to cycle to which the compound of the invention is being administered, including a 28-day cycle. However, the vaginal ring can be inserted for longer or shorter periods of time. See, U.S. Pat. Nos. 5,972,372; 6,126,958; and 6,125,850, which are hereby incorporated by reference, for formulations of the vaginal ring that can be used in the present invention.

The compound of the invention can also be delivered via a transdermal patch. Suitably, use of the patch is timed to the length of the cycle, including a 28-day cycle. However, the patch can remain in place for longer or shorter periods of time.

These compounds can be utilized in methods of contraception, hormone replacement therapy, and the treatment and/or prevention of benign and malignant neoplastic disease, cycle-related symptoms, uterine myometrial fibroids, endometriosis, benign prostatic hypertrophy; carcinomas and adenocarcinomas of the endometrium, ovary, breast, colon, prostate, pituitary, meningioma and other hormone-dependent tumors, dysmenorrheal, dysfunctional uterine bleeding, symptoms of premenstrual syndrome and premenstrual dysphoric disorder, and for inducing amenorrhea. Additional uses of the present progesterone receptor modulators include the synchronization of estrus in livestock. In one embodiment, the neoplastic disease is hormone-dependent.

The compounds of the invention may be administered via a vaginal ring. Suitably, use of the vaginal ring is timed to the 28-day cycle. In one embodiment, the ring is inserted into the vagina, and it remains in place for 3 weeks. During the fourth week, the vaginal ring is removed and menses occurs. The following week a new ring is inserted to be worn another 3 weeks until it is time for the next period. In another embodiment, the vaginal ring is inserted weekly, and is replaced for 3 consecutive weeks. Then, following 1 week without the ring, a new ring is inserted to begin a new regimen. In yet another embodiment, the vaginal ring is inserted for longer or shorter periods of time.

Further, the previously mentioned patch is applied via a suitable adhesive on the skin, where it remains in place for 1 week and is replaced weekly for a total period of 3 weeks. During the fourth week, no patch is applied and menses occurs. The following week a new patch is applied to be worn to begin a new regimen. In yet another embodiment, the patch remains in place for longer, or shorter periods of time.

When used for contraception, the method typically includes delivering a daily dosage unit containing a compound of the invention for 28 consecutive days to a female of childbearing age. Desirably, the method includes delivering the compound over a period of 21 to 27 consecutive days followed by 1 to 7 consecutive days in which no effective amount or no amount of the compound is delivered. Optionally, the period of 1 to 7 days in which no effective amount of the compound is delivered to the subject can involve delivery of a second phase of daily dosage units of 1 to 7 days of a pharmaceutically acceptable placebo. Alternatively, during this “placebo period”, no placebo is administered.

In another embodiment, the method of the invention includes delivering a compound of the invention for 21 consecutive days followed by 7 days in which no effective amount of the compound is delivered. Optionally, during these 7 days, a second phase of 7 daily dosage units of an orally and pharmaceutically acceptable placebo can be delivered. The compound of the invention may optionally be administered in combination with a progestin, antiprogestin, estrogen, or combination thereof.

In a further embodiment, the method of the invention includes delivering a compound of the invention for 23 consecutive days followed by 5 days in which no effective amount of the compound is delivered. Optionally, during these 5 days, a second phase of 5 daily dosage units of an orally and pharmaceutically acceptable placebo can be delivered. The compound of the invention may optionally be administered in combination with a progestin, antiprogestin, estrogen, or combination thereof.

In yet another embodiment, the method of the invention includes delivering a compound of the invention for 25 consecutive days followed by 3 days in which no effective amount of the compound is delivered. Optionally, during these 3 days, a second phase of 3 daily dosage units of an orally and pharmaceutically acceptable placebo can be delivered. The compound of the invention may optionally be administered in combination with a progestin, antiprogestin, estrogen, or combination thereof.

In still a further embodiment, the method of the invention includes delivering a compound of the invention for 27 consecutive days followed by 1 day in which no effective amount of the compound is delivered. Optionally, a second phase of 1 daily dosage unit of an orally and pharmaceutically acceptable placebo can be delivered. The compound of the invention may optionally be administered in combination with a progestin, antiprogestin, estrogen, or combination thereof.

In another embodiment, a method of contraception according to the invention includes administering to a female of childbearing age for 28 consecutive days: (a) a first phase of from 14 to 24 daily dosage units of a progestational agent equal in progestational activity to about 35 to about 100 μg levonorgestrel; (b) a second phase of from 1 to 11 daily dosage units, at a daily dosage of from about 2 to 50 mg, of a compound of the invention, or combination thereof; and (c) optionally, a third phase of daily dosage units of an orally and pharmaceutically acceptable placebo for the remaining days of the 28 consecutive days in which no antiprogestin, progestin or estrogen is administered; wherein the total daily dosage units of the first, second and third phases equals 28.

In yet a further embodiment, a method of contraception includes administering to a female of childbearing age for 28 consecutive days: (a) a first phase of from 14 to 24 daily dosage units of a compound of the invention, or combination thereof; (b) a second phase of from 1 to 11 daily dosage units of an antiprogestin; and (c) optionally, a third phase of daily dosage units of an orally and pharmaceutically acceptable placebo for the remaining days of the 28 consecutive days in which no antiprogestin, progestin or estrogen is administered; wherein the total daily dosage units of the first, second and third phases equals 28.

This invention also includes kits or packages of pharmaceutical formulations designed for use in the regimens described herein. Suitably, the kits contain one or more PR antagonist compounds as described herein.

Advantageously, for use in the kits of the invention, the compound of the invention is formulated for the desired delivery vehicle and route. For example, the compound can be formulated for oral delivery, parenteral delivery, vaginal ring delivery, transdermal delivery, or mucosal delivery, as discussed in detail above. The kit of the invention is preferably a pack (e.g., a blister pack) containing daily doses arranged in the order in which they are to be taken.

In each of the regimens and kits described herein, it is preferred that the daily dosage of each pharmaceutically active component of the regimen remain fixed in each particular phase in which it is administered. It is also understood that the daily dose units described are to be administered in the order described, with the first phase followed in order by the optional phases, including any second and third phases. To help facilitate compliance with each regimen, it is also preferred that the kits contain the placebo described for the final days of the cycle. It is further preferred that each package or kit contain a pharmaceutically acceptable package having indicators for each day of the 28-day cycle, such as a labeled blister package, dial dispenser, or other packages known in the art.

These dosage regimens may be adjusted to provide the optimal therapeutic response. For example, several divided doses of each component may be administered daily or the dose may be proportionally increased or reduced as indicated by the exigencies of the therapeutic situation. In the descriptions herein, reference to a daily dosage unit may also include divided units, which are administered over the course of each day of the cycle contemplated.

In one embodiment, the kit of the invention is designed for daily oral administration over a 28-day cycle, desirably for one oral administration per day, and organized so as to indicate a single oral formulation or combination of oral formulations to be taken on each day of the 28-day cycle. Desirably each kit will include oral tablets to be taken on each of the days specified; desirably one oral tablet will contain each of the combined daily dosages indicated. For example, a kit of the invention can contain 21 to 27 daily dosage units of an effective amount of the compound of the invention and, optionally, 1 to 7 daily dosage units of a placebo and other appropriate components including, e.g., instructions for use.

In another embodiment, the kit of the invention is designed for weekly or monthly administration via a vaginal ring over a 28-day cycle. Suitably, such a kit contains individual packaging for each of the vaginal rings, i.e. one to three, required for a monthly cycle and other appropriate components, including, e.g., instructions for use.

In a further embodiment, the kit of the invention is designed for weekly or monthly administration via a transdermal patch over a 28-day cycle. Suitably, such a kit contains individual packaging for each of the patches, i.e. one to three, required for a monthly cycle and other appropriate components including, e.g., instructions for use.

In still another embodiment, the kit of the invention is designed for parenteral delivery of the compound of the invention. Such a kit is typically designed for delivery at home and may include needles, syringes, and other appropriate packaging and instructions for use.

In yet another embodiment, the kit of the invention contains the compound of the invention in a gel or cream formulation. Optionally, the kit can include appropriate packaging such as a tube or other container, an applicator, and/or instructions for use.

The following examples are illustrative only and are not intended to be a limitation on the present invention.

### EXAMPLE 1

### Culture ARSEF 6144 and its Fermentations

A. Organism:

The strain of Verticillium lecanii, ARSEF 6144, was obtained from the USDA-ARS collection of Entomopathogenic Fungi (ARSEF) at Cornell University, Ithaca, N.Y. It was originally isolated in January, 1999 from a brown citrus aphid, Toxoptera ciricida, of geographic origin in the Dominican Republic (Sabana Grande). For revival and purity check, culture ARSEF 6144 was streaked onto a potato dextrose agar (Difco PDA) plate and grown for up to 21 days at 22° C. The culture grew rapidly having a white surface, which became yellowish and velvety after 14 days. Microscopic morphology at 1000× showed septate hyphae, simple conidiophores branched at several levels and in whorls, elongated phialides with pointed apex, and single celled oval to cylindrical shaped conidia. Culture ARSEF 6144 also grew well and dispersed in potato dextrose broth (Difco PDB) at 22° C. and 200 RPM after 4 days. Culture ARSEF 6144 was stored at −140° C. as a 25% glycerol stock (1.5 ml per cryovial) prepared from a 4-day old liquid culture grown in PDB, and was subcultured onto PDA plates at 22° C. when needed.

A variant of ARSEF 6144 was used for large-scale production of desired compounds in multiple 1 L cultures. This variant, designated ARSEF 6144-A, was chosen on malt-extract agar medium at 22° C. (25 g/L Difco malt extract, 5 g/l Difco peptone, 0.5 g/L Difco yeast extract, 20 g/L Difco agar) as an area of light-yellow cottony growth that lacked pure-white cottony growth. Such areas of raised pure-white aerial growth were associated with lower production of target compounds. Sectors lacking aerial mycelia were also avoided during strain selection.

B. Inoculum Development:

Culture ARSEF 6144 was streaked onto a PDA plate from a frozen vial and incubated at 22° C. for 7 days. One square inch area of the culture lawn was macerated and placed into 50 ml of PDB in a 250 mL Erlenmeyer flask. The liquid seed culture was grown at 22° C. and 200 RPM for 7 days. This single stage seed was used to inoculate liquid and solid fermentations.

C. Shake Flask Fermentation:

Initial shake flask fermentations of culture ARSEF 6144 were performed in PDB in two scales: 100 mL of medium in 500 mL Erlenmeyer flasks and 1 L of medium in 2.8 L Fernbach flasks. Fermentation medium was inoculated with 5% v/v of the single stage seed culture, and incubated at 22° C. and 200 RPM for 7 days. Later fermentations were carried out in production media containing w/w 2% glycerol, 2% sucrose, 0.3% yeast extract, 0.46% ammonium sulfate, and 0.3% calcium carbonate in deionized water.

D. Inoculum Development for Strain ARSEF 6144-A.

Culture ARSEF 6144-A was streaked onto a PDA plate from a frozen vial and incubated at 22° C. for 7 days. An area of culture lawn on PDA of approximately 4 square cm was macerated with a wide bore 10-mL pipet and then used to inoculate 100 mL of potato-dextrose broth (PDB) in a 500 mL Erlenmeyer flask. This first-stage seed culture was grown at 22° C. and 200 rpm for 7 days. Second-stage seed cultures in PDB were inoculated (5% v/v) with this first-stage seed culture. Seed cultures were pooled to prepare a homogenous seed preparation for multiple 1 L fermentations performed in 2.8 L Fernbach flasks.

E. Shake-Flask Fermentation for Large-Scale Production with Strain ARSEF 6144-A:

Multiple 1 L fermentations in 2.8 L Fernbach flasks were performed in production medium consisting of 20 g/L glycerol, 20 g/L sucrose, 3 g/L yeast extract, 4.6 g/L ammonium sulfate, and 3 g/L calcium carbonate in deionized water. Production medium was inoculated with 5% v/v second-stage seed culture. All flasks were incubated at 22° C. and 200 rpm for 7-9 days. Fermentation with this strain was also productive in 500 mL flasks containing 100 mL of the above production medium incubated at 28° C. and 200 rpm for 9 days.

F. Fermentation in Solid Medium:

Initial solid medium fermentations of culture ARSEF 6144 were performed using KenAG brand milk filter paper strips of ⅜×3 inch size, 3 g (moistened with 20 mL of water) sprinkled with coarsely ground Shoprite brand long grain rice, 30 g; in a 500 mL wide mouth Erlenmeyer flask; autoclaved at 121° C. for 20 min. The medium was then supplemented with 22 mL of sterile yeast extract (0.10% w/v) and inoculated with 4 mL (5% v/v) of the single stage seed. Fermentation was carried out at 22° C. for 14 days. Scale-up fermentation was also carried out in 2.8 L Fernbach flasks with higher amounts of the above medium (150 g of ground rice, 15 g of paper strips, 110 mL of water to moisten the paper strips), and 110 mL of sterile 0.1% yeast extract was added after autoclaving. The flask was inoculated with 20 mL (5% v/v) of seed culture and fermented for 14 days at 22° C.

G. Extraction Method:

Wet Diaion™ HP20 resin (50 g per 100 mL medium) was added and stirred for 45 minutes. The cell mass and the resin were collected by centrifugation and extracted with methanol. Solid medium fermentations were extracted twice with sufficient volumes of methanol. Further purification of the ARSEF 6144 components was accomplished by trituration and reversed-phase HPLC as described herein.

### EXAMPLE 2

### Isolation of ARSEF 6144 Components

The isolations of the 4a-hydroxy-4,4,12b,12c-tetramethyl-4a,5,6,6a,7,12,12b,12c-octahydrobenzo[6,7]indeno[1,2-b]indol-3(4H)-one, 4a-hydroxy-4,4,12b,12c-tetramethyl-1,4,4a,5,6,6a,7,12,12b,12c-decahydrobenzo[6,7] indeno[1,2-b]indol-3(2H)-one, 4aS,6aR,12bR,12cR)-4,4,12b,12c-tetramethyl-3-oxo-3,4,4a,5,6,6a,7,12,12b,12c-decahydrobenzo[6,7] indeno[1,2-b]indol-10-yl 4-O-methyl-α-D-glucopyranoside and (3S,4aR,6aR,12bR,12cS)-4,4,12b,12c-tetramethyl-1,3,4,5,6,6a,7,12,12b,12c-decahydrobenzo[6,7] indeno[1,2-b]indole-3,4a(2H)-diol were accomplished by multiple reversed-phase column chromatography of fermentation extracts as described.

The whole solid fermentation was soaked in methanol and was either filtered or centrifuged to provide a particulate-free extract. A 10% volume of washed Diaion™ HP20 resin was slurried with the liquid fermentation and then filtered or centrifuged. The supernatant was discarded and the pellet was soaked in methanol. The methanol extract was then filtered or centrifuged and combined with the solid fermentation methanol extract. The combined extracts were dried in vacuo and the solids were triturated with 500 mL of hexanes. The hexanes extract was decanted and dried in vacuo to yield 692.2 mg of residue. The hexanes insoluble solids were dried in vacuo to remove remaining traces of hexanes to give 2.82 g of material. This crude extract was chromatographed on a YMC™ 250×50 mm ODS-A column using a gradient mobile phase from 5% to 95% solvent B (0.05% trifluoroacetic acid in acetonitrile) in solvent A (0.05% trifluoroacetic acid in water) over 120 minutes at a flow rate of 20 mL per minute and kept at that flow rate, at 95% solvent B in solvent A for another 20 minutes. Ultraviolet absorbance is monitored at 254 nm while 40 mL fractions were collected every 2 minutes.

### A. 4a-hydroxy-4,4,12b,12c-tetramethyl-4a,5,6,6a,7,12,12b,12c-octahydrobenzo[6,7]indeno[1,2-b]indol-3(4H)-one

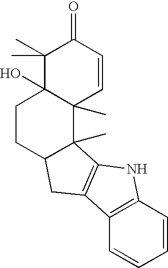

### 4a-hydroxy-4,4,12b,12c-tetramethyl-4a,5,6,6a,7,12,12b,12c-octahydrobenzo[6,7] indeno[1,2-b]indol-3(4H)-one

4a-hydroxy-4,4,12b,12c-tetramethyl-4a,5,6,6a,7,12,12b,12c-octahydrobenzo[6,7] indeno[1,2-b]indol-3(4H)-one and 4a-hydroxy-4,4,12b,12c-tetramethyl-1,4,4a, 5,6,6a,7,12,12b,12c-decahydrobenzo[6,7]indeno[1,2-b]indol-3(2H)-one were isolated by combining fractions 57 to 60, collected between 112 and 120 minutes, and reducing to 16 mg of residue in vacuo. The residue was further chromatographed on a YMC™ 250×10 mm ODS-A column with a gradient mobile phase from 50% to 70% solvent B (0.05% trifluoroacetic acid in methanol) in solvent A (0.05% trifluoroacetic acid in water) over 60 minutes at a flow rate of 2 mL per minute. Ultraviolet absorbance was monitored at 240 nm while 2 mL fractions were collected each minute. Fractions 41 and 42, collected between 40 and 42 minutes were combined and evaporated in vacuo to a residue of 1.9 mg of 4a-hydroxy-4,4,12b,12c-tetramethyl-4a,5,6,6a,7,12,12b,12c-octahydrobenzo[6,7]indeno[1,2-b]indol-3(4H)-one.

By using the procedure described above, 4a-hydroxy-4,4,12b,12c-tetramethyl-4a,5,6,6a,7,12,12b,12c-octahydrobenzo[6,7]indeno[1,2-b]indol-3(4H)-one was isolated as an off-white powder.
a) Molecular Formula: C₂₃H₂₇NO₂
b) Molecular Weight: high resolution positive ion electrospray MS m/z=350.21161 (M+H)⁺; high resolution negative ion electrospray MS m/z=348.19630 (M−H)⁻
c) Ultraviolet Absorption Spectrum: λₘₐₓ nm (acetonitrile-water-formic acid)=234, 284
d) Proton NMR (400 MHz; δ, CDCl₃): 7.92, 7.49, 7.35, 7.31, 7.12, 7.11, 6.18, 2.85, 2.77, 2.43, 2.07, 1.96, 1.84, 1.80, 1.51, 1.40, 1.32, 1.25.
e) Carbon NMR (100 MHz; δ, CDCl₃): 203.9, 155.2, 152.3, 140.3, 128.4, 125.4, 121.1, 120.1, 118.9, 118.1, 111.8, 80.4, 53.4, 52.3, 49.6, 48.2, 29.7, 28.4, 27.5, 25.6, 23.7, 21.4, 19.8.

### B. 4a-hydroxy-4,4,12b,12c-tetramethyl-1,4,4a,5,6,6a,7,12,12b,12c-decahydrobenzo[6,7]indeno[1,2-b]indol-3(2H)-one

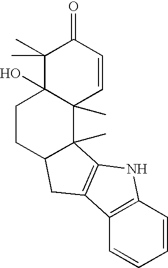

### 4a-hydroxy-4,4,12b,12c-tetramethyl-1,4,4a,5,6,6a,7,12,12b,12c-decahydrobenzo[6,7]indeno[1,2-b]indol-3(2H)-one

Using the procedure described above, Fractions 45 and 46, collected between 44 and 46 minutes were combined and evaporated in vacuo to a residue of 2.0 mg of 4a-hydroxy-4,4,12b,12c-tetramethyl-1,4,4a,5,6,6a,7,12,12b,12c-decahydrobenzo[6,7]indeno[1,2-b]indol-3(2H)-one. 4a-hydroxy-4,4,12b,12c-tetramethyl-1,4,4a,5,6,6a,7,12,12b,12c-decahydrobenzo[6,7] indeno[1,2-b]indol-3(2H)-one was isolated as an off-white powder.
a) Molecular Formula: C₂₃H₂₉NO₂
b) Molecular Weight: high resolution positive ion electrospray MS m/z=352.22700 (M+H)⁺; high resolution negative ion electrospray MS m/z=350.21242 (M−H)⁻
c) Ultraviolet Absorption Spectrum: λₘₐₓ nm (acetonitrile-water-formic acid)=232, 284
d) Proton NMR (400 MHz; δ, CHCl₃): 7.73, 7.44, 7.31, 7.09, 7.08, 2.92, 2.82, 2.75, 2.74, 2.72, 2.42, 2.03, 1.97, 1.73, 1.65, 1.63, 1.34, 1.27, 1.25, 1.13.
e) Carbon NMR (100 MHz; δ, CDCl₃): 216.8, 152.7, 140.2, 125.4, 120.8, 119.9, 118.8, 117.4, 111.7, 81.5, 54.9, 53.3, 49.5, 43.7, 34.7, 32.5, 29.9, 27.7, 24.5, 23.1, 22.6, 21.3, 17.0.

### C. 4aS,6aR,12bR,12cR)-4,4,12b,12c-tetramethyl-3-oxo-3,4,4a,5,6,6a,7,12,12b,12c-decahydrobenzo[6,7]indeno[1,2-b]indol-10-yl 4-O-methyl-α-D-glucopyranoside

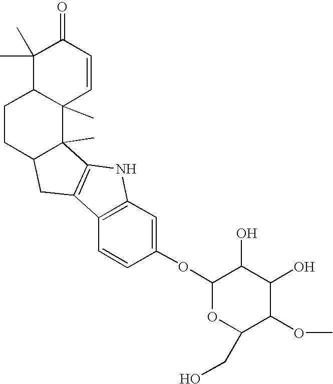

### (4aS,6aR,12bR,12cR)-4,4,12b,12c-tetramethyl-3-oxo-3,4,4a,5,6,6a,7,12,12b,12c-decahydrobenzo[6,7]indeno[1,2-b]indol-10-yl 4-O-methyl-α-D-glucopyranoside

4aS,6aR,12bR,12cR)-4,4,12b,12c-tetramethyl-3-oxo-3,4,4a,5,6,6a,7,12,12b,12c-decahydrobenzo[6,7]indeno[1,2-b]indol-10-yl 4-O-methyl-α-D-glucopyranoside was isolated by combining fractions 44 to 46, collected between 88 and 92 minutes during the first 250×50 mm ODS-A chromatography of the crude extract, and reduced to 5 mg of residue in vacuo. The residue was further chromatographed on an YMC™ 250×10 mm ODS-A column with a gradient mobile phase from 60% to 100% methanol in water over 60 minutes at a flow rate of 2 mL per minute. Ultraviolet absorbance was monitored at 240 nm while 2 mL fractions were collected each minute. Fractions 36 and 37, collected between 35 and 37 minutes, were combined and evaporated in vacuo to a residue of 0.5 mg of (4aS,6aR,12bR,12cR)-4,4,12b,12c-tetramethyl-3-oxo-3,4,4a,5,6,6a,7,12,12b,12c-decahydrobenzo[6,7]indeno[1,2-b]indol-10-yl 4-O-methyl-α-D-glucopyran.

By using the procedure described herein, (4aS,6aR,12bR,12cR)-4,4,12b,12c-tetramethyl-3-oxo-3,4,4a,5,6,6a,7,12,12b,12c-decahydrobenzo[6,7] indeno[1,2-b]indol-10-yl 4-O-methyl-α-D-glucopyran was isolated as a pale pink powder.

a) Molecular Formula: C₃₀H₃₉NO₇;

b) Molecular Weight: positive ion electrospray MS m/z=526.2 (M+H)⁺; high resolution FTMS m/z=525.26514 (M)⁺;

c) Ultraviolet Absorption Spectrum: λₘₐₓ nm (acetonitrile-water-formic acid)=236, 278, 300;

d) Proton NMR (400 MHz; δ, CDCl₃): 8.01, 7.33, 7.24, 7.07, 6.86, 6.03, 4.88, 3.94, 3.77, 3.75, 3.68, 3.62, 3.44, 3.30, 2.76, 2.31, 1.87, 1.80, 1.71, 1.56, 1.32, 1.26, 1.22, 1.12, 1.07.

### D. (3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol

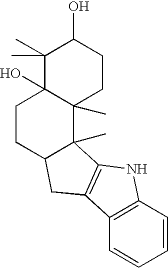

### (3S,4aR,6aR,12bR,12cS)-4,4,12b,12c-tetramethyl-1,3,4,5,6,6a,7,12,12b,12c-decahydrobenzo[6,7] indeno[1,2-b]indole-3,4a(2H)-diol

(3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol was isolated from the hexanes wash of the crude fermentation extract. The material was adsorbed onto a 2.5×120 cm column (680 mL volume) of Sephadex™ LH-20 and then eluted with methanol while collecting 40 mL per 120-minute fractions. The fractions were analyzed by HPLC. Fractions 13 to 16 were combined and reduced in vacuo to a residue of 55.5 mg. This was further purified by reverse phase chromatography using an YMC™ 250×20 mm ODS-A column with a gradient mobile phase from 60% to 100% methanol in water over 60 minutes at a flow rate of 8 mL per minute. Ultraviolet absorbance was monitored at 254 nm while 8 mL fractions were collected each minute. Fractions 48 and 49, collected between 47 and 50 minutes, were combined and evaporated in vacuo to a residue of 1.4 mg of (3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol.

(3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol was also made by reduction of 4a-hydroxy-4,4,12b,12c-tetramethyl-1,4,4a,5,6,6a,7,12,12b,12c-decahydrobenzo[6,7]indeno[1,2-b]indol-3(2H)-one as follows. To a solution of 4a-hydroxy-4,4,12b,12c-tetramethyl-1,4,4a,5,6,6a,7,12,12b,12c-decahydrobenzo[6,7]indeno[1,2-b]indol-3(2H)-one (2.5 mg, 7.1 μm, in 5 mL of absolute ethanol) was added 37 molar equivalents (10 mg, 0.26 mmol) of sodium borohydride. The solution was stirred at room temperature for two hours. The reaction was monitored by LCMS, checking for the reduction product. The solution was neutralized with 2 mL of acetone and dried under a stream of nitrogen, flushing with methanol and drying three times to remove excess sodium borohydride. The product was chromatographed on an YMC™ 250×10 millimeter ODS-A column with a gradient mobile phase from 50% to 70% solvent B (0.05% trifluoroacetic acid in acetonitrile) in solvent A (0.05% trifluoroacetic acid in water) over 60 minutes at a flow rate of 2 mL per minute. Ultraviolet absorbance was monitored at 254 nanometers while 2 mL fractions were collected every minute. Fractions 36 to 37, collected between 35 and 37 minutes, were combined and dried in vacuo to a residue of 1.0 mg ((3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol). Fractions 54 and 55, collected between 53 and 55 minutes, were combined and dried in vacuo to a residue of 0.7 mg (isomer of (3S,4aR,6aR,12bR,12cS)-4,4,12b,12c-tetramethyl-1,3,4,5,6,6a,7,12,12b,12c-decahydrobenzo[6,7]indeno[1,2-b]indole-3,4a(2H)-diol).

By using the procedure described above, (3S,4aR,6aR,12bR,12cS)-4,4,12b,12c-tetramethyl-1,3,4,5,6,6a,7,12,12b,12c-decahydrobenzo[6,7] indeno[1,2-b]indole-3,4a(2H)-diol was isolated as an off-white powder.

a) Molecular Formula: C₂₃H₃₁NO₂;

b) Molecular Weight: high resolution ESI FTMS m/z=354.24264 (M+H)⁺; high resolution ESI FTMS m/z=352.22817 (M−H)⁻;

c) Ultraviolet Absorption Spectrum: λₘₐₓ nm (acetonitrile-water-formic acid)=234, 284;

d) Proton NMR (400 MHz; δ, CDCl₃): 7.72, 7.43, 7.30, 7.08, 7.07, 3.62, 2.84, 2.69, 2.66, 2.38, 2.30, 2.00, 1.87, 1.74, 1.72, 1.68, 1.38, 1.25, 1.24, 1.14, 1.03.

e) Carbon NMR (100 MHz; δ, CDCl₃): 154.3, 139.9, 125.6, 120.4, 119.7, 119.6, 117.3, 111.6, 81.5, 78.5, 54.9, 49.6, 43.9, 42.4, 30.5, 27.7, 26.7, 25.3, 24.8, 24.4, 23.4, 21.9, 17.0.

### EXAMPLE 3

### Characterization of Compounds

A. PRE Luciferase Reporter Assay to Determine Progestin Activity:

Progestin activity was measured using a common luciferase reporter system in cells expressing endogenous progesterone receptors (PR). T47D breast cancer cells were plated at 50,000 cells/well in 96 well plates in DMEM/F-12 supplemented with penicillin/streptomycin, Glutamax and 5% FBS (Gibco). Cells were infected with a 1:1000 dilution of an adenovirus vector expressing luciferase under the control of three copies of a consensus progesterone receptor regulatory element (PRE) located in the promoter region. Following a two-hour post-infection recovery, cells were treated for 20 hours with progesterone (P4) or test compounds. Luciferase reporter activity was quantified using the Luciferase Assay System (Promega) and a Victor2 luminometer (Perkin Elmer).

B. Quantigene Branched DNA Assay to Determine PR-isoform Selectivity.

T47D cells were plated as in the PRE assay and treated with compound for 15 hours. Expression of tissue factor (TF), a PR-β isoform regulated gene, or periplakin (PPL), a PR-A regulated gene, was measured by the Quantigene branched DNA assay (Genospectra) using the Victor2 luminometer following the manufacturer's recommended protocol.

C. Statistical Analysis:

EC₅₀ values were determined using the dose response nonlinear fit model from the SAS statistics package (SAS Institute Inc). Efficacies were calculated as a percentage of the maximum P4 response.

4a-hydroxy-4,4,12b,12c-tetramethyl-4a,5,6,6a,7,12,12b,12c-octahydrobenzo[6,7] indeno[1,2-b]indol-3(4H)-one and 4aS,6aR,12bR,12cR)-4,4,12b,12c-tetramethyl-3-oxo-3,4,4a,5,6,6a,7,12,12b,12c-decahydrobenzo[6,7]indeno[1,2-b]indol-10-yl 4-O-methyl-α-D-glucopyranoside efficacies were <1%;

4a-hydroxy-4,4,12b,12c-tetramethyl-1,4,4a,5,6,6a,7,12,12b,12c-decahydrobenzo[6,7]indeno[1,2-b]indol-3(2H)-one efficacies were <1 to 2%;

(3S,4aR,6aR,12bR,12cS)-4,4,12b,12c-tetramethyl-1,3,4,5,6,6a,7,12,12b,12c-decahydrobenzo[6,7] indeno[1,2-b]indole-3,4a(2H)-diol efficacies were 68% to 97% and EC₅₀'s were 7.3 to 33 nM.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A compound having the structure: 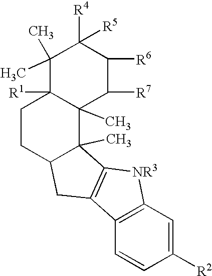
R¹ is H or OH;
R² is H or 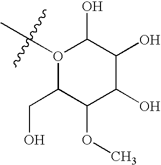
R³ is H or CH₃;
R⁴, R⁵ is ═O, OH;
R⁶, R⁷ is CH═CH, CH₂—CH₂, OH, and an epoxide,
or a pharmaceutically acceptable salt, ester, or ether thereof.

2. A compound according to claim 1, selected from the group consisting of: 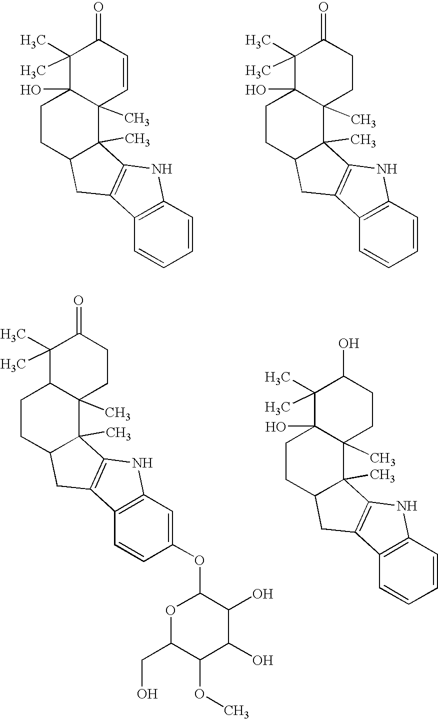
or a pharmaceutically acceptable salt or ester thereof.

3. A compound having the characteristics:
(a) molecular formula C₂₃H₂₇NO₂;
(b) a molecular weight characterized by a high resolution ion electrospray MS m/z of 350.21161 (M+H)+ and a negative ion electrospray MS m/z of 348.2; and
(c) an ultraviolet absorption spectrum of λₘₐₓ nm
(acetonitrile/water/formic acid) of 234, 284.

4. The compound of claim 3 further characterized by
a proton nuclear magnetic resonance (400 MHz; δ, CDCl₃): 7.92, 7.49, 7.35, 7.31, 7.12, 7.11, 6.18, 2.85, 2.77, 2.43, 2.07, 1.96, 1.84, 1.80, 1.51, 1.40, 1.32, 1.25; and
a carbon nuclear magnetic resonance (100 MZ; δ, CDCl₃): 203.9, 155.2, 152.3, 140.3, 128.4, 125.4, 121.1, 118.9, 118.1, 111.8, 80.4, 53.4, 52.3, 49.6, 48.2, 29.7, 28.4, 27.5, 25.6, 23.7, 21.4, 19.8.

5. The compound of claim 3, which is 4a-hydroxy-4,4,12b,12c-tetramethyl-4a,5,6,6a,7,12,12b,12c-octahydrobenzo[6,7]indeno[1,2-b]indol-3(4H)-one.

6. A compound having the characteristics:
(a) a molecular formula: C₂₃H₂₉NO₂;
(b) a molecular weight characterized by a high resolution positive ion electrospray MS m/z=352.22700 (M+H)⁺ and a high resolution negative ion electrospray MS m/z=350.21242 (M−H)⁻; and
(c) an ultraviolet absorption spectrum of λₘₐₓ nm (acetonitrile-water-formic acid)=232, 284.

7. The compound according to claim 6 further characterized by
a proton nuclear magnetic resonance of (400 MHz; δ, CHCl₃):
7.73, 7.44, 7.31, 7.09, 7.08, 2.92, 2.82, 2.75, 2.74, 2.72, 2.42, 2.03, 1.97, 1.73, 1.65, 1.63, 1.34, 1.27, 1.25, 1.13; and
a carbon nuclear magnetic resonance of (100 MHz; δ, CDCl₃): 216.8, 152.7, 140.2, 125.4, 120.8, 119.9, 118.8, 117.4, 111.7, 81.5, 54.9, 53.3, 49.5, 43.7, 34.7, 32.5, 29.9, 27.7, 24.5, 23.1, 22.6, 21.3, 17.0.

8. The compound according to claim 6 which is 4a-hydroxy-4,4,12b,12c-tetramethyl-1,4,4a,5,6,6a,7,12,12b,12c-decahydrobenzo[6,7]indeno[1,2-b]indol-3(2H)-one.

9. A compound having the characteristics:
(a) a molecular formula of C₃₀H₃₉NO₇;
(b) a molecular weight of positive ion electrospray MS m/z=526.2 (M+H)⁺; negative ion electrospray MS m/z=524.9 (M−H)⁻; and
(c) an ultraviolet absorption spectrum of λₘₐₓ nm (acetonitrile-water-formic acid)=236, 278, 300.

10. The compound according to claim 9, further characterized by
a proton nuclear magnetic resonance (400 MHz; δ, CDCl₃): 8.01, 7.33, 7.24, 7.07, 6.86, 6.03, 4.88, 3.94, 3.77, 3.75, 3.68, 3.62, 3.44, 3.30, 2.76, 2.31, 1.87, 1.80, 1.71, 1.56, 1.32, 1.26, 1.22, 1.12, 1.07.

11. The compound according to claim 9, which is (4aS,6aR,12bR,12cR)-4,4,12b,12c-tetramethyl-3-oxo-3,4,4a,5,6,6a,7,12,12b,12c-decahydrobenzo[6,7]indeno[1,2-b]indol-10-yl 4-O-methyl-α-D-glucopyranoside.

12. A compound characterized by
(a) a molecular formula of C₂₃H₃₁NO₂;
(b) a molecular weight characterized by a high resolution ESI FTMS m/z=354.24264 (M+H)⁺; high resolution ESI FTMS m/z=352.22817 (M−H)⁻; and
(c) an ultraviolet absorption spectrum of λₘₐₓ nm (acetonitrile-water-formic acid)=234, 284.

13. The compound according to claim 12, further characterized by a proton nuclear magnetic resonance (400 MHz; δ, CDCl₃):
7.72, 7.43, 7.30, 7.08, 7.07, 3.62, 2.84, 2.69, 2.66, 2.38, 2.30, 2.00, 1.87, 1.74, 1.72, 1.68, 1.38, 1.25, 1.24, 1.14, 1.03; and
a carbon nuclear magnetic resonance (100 MHz; δ, CDCl₃): 154.3, 139.9, 125.6, 120.4, 119.7, 119.6, 117.3, 111.6, 81.5, 78.5, 54.9, 49.6, 43.9, 42.4, 30.5, 27.7, 26.7, 25.3, 24.8, 24.4, 23.4, 21.9, 17.0.

14. The compound according to claim 12, which is (3S,4aR,6aR,12bR,12cS)-4,4,12b,12c-tetramethyl-1,3,4,5,6,6a,7,12,12b,12c-decahydrobenzo[6,7] indeno[1,2-b]indole-3,4a(2H)-diol.

15. A process for the preparation of a compound of claim 1 which comprises culturing a strain of Verticillium lecanii capable of producing the compound and isolating the compound.

16. The process according to claim 1, wherein the compound is isolated by multiple reversed-phased column chromatography of fermentation extracts prepared by soaking in methanol.

17. An isolated culture of Verticillium lecanii strain ARSEF 6144-A, assigned Agricultural Research Service Culture Collection (NRRL) Accession No. NRRL 30907.

18. A progesterone receptor modulator isolated from the culture of claim 17.

19. A method of producing a progesterone receptor modulator compound comprising the step of culturing Verticillium lecanii strain ARSEF 6144-A under conditions suitable to produce the compound.

20. A progesterone receptor modulator composition comprising a compound according to claim 1, or a pharmaceutically acceptable salt thereof, and a pharmaceutically acceptable carrier or excipient.

21. A method of inducing contraception, providing hormone replacement therapy, treating cycle-related symptoms, and treating or preventing benign or malignant neoplastic disease comprising administering to a mammal in need thereof a pharmaceutically effective amount of a compound of claim 1.

22. The method according to claim 21 wherein said cycle-related symptoms are psychological.

23. The method according to claim 22, wherein said psychological symptoms include mood changes, irritability, anxiety, lack of concentration, or decrease in sexual desire.

24. The method according to claim 21, wherein said cycle-related symptoms are physical.

25. The method according to claim 24, wherein said physical symptoms include breast tenderness, bloating, fatigue, cramping, irregular menstrual bleeding, or food cravings.

26. The method according to claim 21, wherein the hormone-dependent neoplastic disease is selected from the group consisting of uterine fibroids, endometriosis, benign prostatic hypertrophy; carcinomas and adenocarcinomas of the endometrium, ovary, breast, colon, prostate, pituitary, and meningioma.

27. A method of treating neurodegenerative disorders comprising administering to a mammal in need thereof a pharmaceutically effective amount of a compound of claim 1.

28. The method according to claim 22, wherein the neurodegenerative disorders are selected from the group consisting of Alzheimer's disease, Parkinson's disease, and neuronal damage following ischemia or trauma.
